(19) United States
(12) Patent Application Publication
(10) Pub. No.: US 2026/0165953 A1
(43) Pub. Date: Jun. 18, 2026

(54) THE HIPPO PATHWAY AS A PHARMACOLOGIC TARGET FOR TREATING REFRACTIVE DISORDERS

(71) Applicants: Centre for Eye and Vision Research Limited, Hong Kong (HK); The Hong Kong Polytechnic University, Hong Kong (HK)

(72) Inventors: Chuen LAM, Hong Kong (HK); Yan Yin TSE, Hong Kong (HK); Jia Jun WANG, Hong Kong (HK); Jing Fang BIAN, Hong Kong (HK); Bing ZUO, Hong Kong (HK); Chi Ho TO, Hong Kong (HK)

(21) Appl. No.: 19/127,421

(22) PCT Filed: Nov. 21, 2023
(86) PCT No.: PCT/CN2023/132931

§ 371 Date: May 6, 2025

Related U.S. Application Data
(60) Provisional application No. 63/427,069, filed on Nov. 21, 2022.

(51) Int. Cl. A61K 9/00; A61K 31/4184; A61K 31/485; A61K 31/4985; A61K 31/519; A61K 45/06

(52) U.S. Cl. CPC A61K 9/0019; A61K 31/4184; A61K 31/485; A61K 31/4985; A61K 31/519; A61K 45/06; A61K 2300/00

(57) ABSTRACT

A novel pharmacologic treatment of refractive disorders including myopia and hyperopia is provided. The method of treating refractive disorders including myopia and hyperopia is mainly through targeting the Hippo biological pathway using agonists, antagonists or gene therapy targeting the HIPPO signaling, including MST1/MST2, LATS1/LATS2 kinases, and YAP/TAZ proteins. It is also found that the drug XMU-MP-1, a reversible and selective inhibitor of MST1/2, is a promising drug to target the Hippo biological pathway.

### CROSS-REFERENCE TO RELATED APPLICATIONS

This application is a 371 application of the International patent application number PCT/CN2023/132931 filed on Nov. 21, 2023, which claims priority from U.S. provisional patent application No. 63/427,069 filed on Nov. 21, 2022, and the disclosure of which are incorporated herein by reference in its entirety.

### FIELD OF THE INVENTION

The present invention generally relates to the field of ophthalmology. More specifically the present invention relates to a particular focus on addressing refractive disorders.

### BACKGROUND OF THE INVENTION

Myopia is a common ocular disorder with a global prevalence of around 30%, projected to increase to 50% by 2050. Eyes with high myopia face an elevated risk of severe conditions like myopic maculopathy, retinal detachment, and glaucoma. Therefore, a range of treatments exists to slow myopia progression in children, falling into two main categories: optical treatments, including specialized lenses, and pharmaceutical treatments, primarily using atropine eye drops.

In mainland China, atropine eye drops, particularly in low doses (0.01%-0.05%), hold a substantial share in the myopia management market, with estimates ranging from 40-50%. However, atropine's application is limited due to side effects like photophobia, blurred near vision, allergic conjunctivitis, and systemic effects. Experimental models inducing myopia involve the use of negative lenses, causing hyperopic defocus and triggering biochemical cascades that accelerate axial length (AL) elongation. The regulation of eye growth is considered a homeostatic process determined by the balance between “Growth” and “STOP” signals, both optically and biochemically. Consequently, the biochemical signals are believed to originate from the retina and culminate in the sclera. However, the exact molecular mechanisms behind myopia are still unclear, and as of now, there is no FDA or cFDA-approved drug for clinical use. The need for an alternative pharmaceutical candidate with comparable effectiveness but lower toxicity and fewer side effects is crucial.

To address the challenges posed by atropine, there is an ongoing quest for alternatives in the pharmaceutical field. The mode of action of atropine against myopia remains unclear, and its application is restricted due to toxicity concerns at higher doses. Regulatory bodies like the cFDA permit only the lowest dosage under strict regulations. The search for alternative pharmaceutical candidates aims to find options that are as effective as atropine but come with lower toxicity and fewer side effects, providing a potential solution to the existing clinical gap in myopia management.

Therefore, the present invention addresses this need.

### SUMMARY OF THE INVENTION

It is an objective of the present invention to provide a method, a composition, or a use to solve the aforementioned technical problems.

In accordance with a first aspect of the present invention, a method of treating a refractive disorder is provided. Specifically, the method includes administering an agonist, an antagonist or a gene therapy selectively activates or inactivates the HIPPO signaling pathway, a signaling pathway influences cellular proliferation, organ growth and organ regeneration.

In accordance with one embodiment of the present invention, the refractive disorder includes myopia and hyperopia.

In accordance with one embodiment of the present invention, the agonist, the antagonist or the gene therapy specifically targets MST1/MST2 kinase, LATS1/LATS2 kinase or YAP/TAZ protein.

In accordance with one embodiment of the present invention, the agonist includes XMU-MP-1, a pharmaceutically acceptable salt thereof or a derivative compound thereof.

In accordance with one embodiment of the present invention, the XMU-MP-1 or the derivative compound thereof inhibits MST1/2 and activates the Hippo pathway.

In accordance with one embodiment of the present invention, the XMU-MP-1 or the derivative compound thereof is in a dosage of 2 nmol to 200 nmol.

In accordance with one embodiment of the present invention, the administration is through the cornea and/or the blood-retinal barrier and reach the retina.

In accordance with one embodiment of the present invention, the administration is conducted in various suitable forms, including an injection form, an eye drop form, an eye ointment form, a hydrogel form, an ultrasonic ocular drug delivery form, a nanoparticle form, a microemulsion drug delivery form, and a drug-loaded contact lenses form. The administration can also be integrated with other delivery systems, such as ultrasonic ocular drug delivery systems, drug-loaded contact lenses, and hydrogel.

In accordance with a second aspect of the present invention, a pharmaceutical composition including an agonist, an antagonist or a gene therapy selectively activating or inactivating the HIPPO signaling pathway, a signaling pathway influences cellular proliferation, organ growth and organ regeneration, for treating a refractive disorder in a subject in need thereof is provided, particularly, the composition further comprises a pharmaceutically acceptable addition.

In accordance with one embodiment of the present invention, the refractive disorder includes myopia and hyperopia.

In accordance with one embodiment of the present invention, the agonist, the antagonist or the gene therapy specifically targets MST1/MST2 kinase, LATS1/LATS2 kinase or YAP/TAZ protein.

In accordance with one embodiment of the present invention, the agonist includes XMU-MP-1, a pharmaceutically acceptable salt thereof or a derivative compound thereof.

In accordance with one embodiment of the present invention, the XMU-MP-1 or the derivative compound thereof inhibits MST1/2 and activates the Hippo pathway.

In accordance with one embodiment of the present invention, the XMU-MP-1 or the derivative compound thereof is in a dosage of 2 nmol to 200 nmol.

In accordance with one embodiment of the present invention, the composition is formulated to an administration form of that enables delivery to the patient's retina through the cornea and/or the blood-retinal barrier.

In accordance with one embodiment of the present invention, the administration form is selected from an immediate-release form or a controlled-release form.

In accordance with one embodiment of the present invention, the administration form includes an injection form, an eye drop form, an eye ointment form, a hydrogel form, an ultrasonic ocular drug delivery form, a nanoparticle form, a microemulsion drug delivery form, and a drug-loaded contact lenses form.

In accordance with one embodiment of the present invention, the pharmaceutically acceptable addition includes an excipient, a stability additive, a carrier, a diluent, and a solubilizer.

In accordance with a third aspect of the present invention, a usage of a pharmaceutical composition including an agonist, an antagonist or a gene therapy selectively activating or inactivating the HIPPO signaling pathway, a signaling pathway influences cellular proliferation, organ growth and organ regeneration, for treating a refractive disorder in a subject in need thereof is provided.

In accordance with one embodiment of the present invention, the refractive disorder is selected from myopia or hyperopia.

In accordance with one embodiment of the present invention, the agonist, the antagonist or the gene therapy specifically targets MST1/MST2 kinase, LATS1/LATS2 kinase or YAP/TAZ protein.

In accordance with one embodiment of the present invention, the agonist includes XMU-MP-1, a pharmaceutically acceptable salt thereof or a derivative compound thereof.

In accordance with one embodiment of the present invention, the agonist the XMU-MP-1 or the derivative compound thereof inhibits MST1/2 and activates the Hippo pathway.

In accordance with one embodiment of the present invention, the XMU-MP-1 or the derivative compound thereof is in a dosage of 2 nmol to 200 nmol.

### BRIEF DESCRIPTION OF THE DRAWINGS

Embodiments of the invention are described in more details hereinafter with reference to the drawings, in which:

FIG. 1 depicts protein identification, quantification, and Gene ontology (GO) function classification analysis using the SWATH-MS approach;

FIG. 2 depicts HIPPO signaling is enriched as a significant pathway involved with a predicted activity pattern after B-H Multiple Testing Correction using IPA;

FIGS. 3A-3B show the workflow of r MRM-based targeted proteomics for confirming changes of individual proteins associated with Hippo pathway, in which FIG. 3A depict the workflow and FIG. 3B demonstrates the calculations; and

FIGS. 4A-4D shows the effect of intravitreally injected XMU-MP-1 on various parameters, such as refractive error (FIG. 4A), vitreous chamber depth (FIG. 4B), lens thickness (FIG. 4C), and choroidal thickness (FIG. 4D) in unilateral FDM.

### DETAILED DESCRIPTION

In the following description, methods, compositions, usages of treating a refractive disorder and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

HIPPO is a signaling pathway that influences cellular proliferation and destiny, potentially impacting the growth and regeneration of organs. It may stimulate the activation of LATS kinases, which modulate gene expression by suppressing the function of transcriptional co-activator proteins in mammals (e.g., YAP and TAZ). A detailed description of the HIIPO signaling pathway is found in Misra et al., Annu Rev Genet. 2018 Nov. 23; 52: 65-87, the disclosure of which is incorporated herein by reference. Other literature extensively underscores the fundamental roles of the HIPPO pathway in organ growth control, stem cell function, regeneration, and tumor suppression (Harvey et al., 2013, Yu et al., 2015, Wang et al., 2017, Maugeri-Sacca and De Maria, 2018, the disclosures of which are incorporated by reference herein).

The regulation of eye growth is posited as a homeostatic process hinging on the equilibrium between “Growth” and “STOP” signals, both optically and biochemically (Wallman, 1990). The former signals accelerate axial length (AL) elongation, while the latter halts AL elongation. In animal models, myopia induction involves affixing a negative lens to the eyes, causing the image to focus behind the retina and imposing an optical Growth signal (hyperopic defocus). This initiates a cascade of biochemical Growth signals, elevating the AL elongation rate. During recovery after removing the negative lens, exposure to an image focused in front of the retina imposes an optical STOP signal (myopic defocus), triggering a cascade of biochemical STOP signals (Guo et al., 2019, Zhou et al., 2018b, Li et al., 2016a, Chun et al., 2011, the disclosure of which is incorporated by reference herein).

Moreover, it is widely accepted that these biochemical signal cascades originate at the retina and culminate in the sclera. Despite these understandings, the precise molecular mechanism driving myopia remains elusive. Presently, there is no FDA-approved or cFDA-endorsed drug for clinical use in this context. The intricate interplay of optical and biochemical signals and their convergence at the retina and sclera highlights the complexities of myopia regulation that necessitate further research and potential therapeutic interventions.

In recent years, proteomic approaches have proven to be potent instruments capable of simultaneous screening of thousands of protein candidates, allowing for the comprehensive detection of global protein expression regulation. Within discovery-based proteomics, the SWATH-MS-based approach has gained prominence as a valuable platform for biomarker discovery and a deeper comprehension of biological mechanisms. Employing a proteomics approach, the present invention provides a novel proteomic pathway identification methodology that is used to create pharmacological products for controlling ocular growth by gene therapy.

The present invention uses the connection of the HIPPO pathway at the protein level through the demonstration of altered ocular growth through targeted modulation of the HIPPO pathway in the context of myopia. Previous research on the Hippo pathway predominantly focused on its role in cancer, with synthetic drugs designed for cancer treatment. The present invention demonstrates the involvement of the HIPPO pathway and its associated proteins in ongoing myopia, utilizing a Lens-induced myopia (LIM) animal model. In particular, the present invention determines that five differentially expressed proteins are involved in myopia, demonstrating their pharmacological targeting of the HIPPO pathway to modulate eye growth. By using the HIPPO pathway to control myopia, therapeutic interventions are developed.

Recognized as a reversible and selective MST1/2 inhibitor activating the downstream effector Yes-associated protein (YAP), the benzenesulfonamide XMU-MP-1 has pharmacological implications. The present invention demonstrates that intravitreal injection of XMU-MP1 robustly inhibits lens-induced myopia. This substantiates the HIPPO pathway as a molecular target for pharmacological intervention in ocular growth modulation, thereby treating refractive disorders such as myopia and hyperopia. Importantly, XMU-MP1 demonstrates a high level of efficacy and exhibits no discernible signs of toxicity in animal models.

In accordance with a first aspect of the present invention, the present invention introduces a method that involves the administration of agents targeting the HIPPO signaling pathway. Because this pathway may be used to control organ growth, stem cell function, and tumor suppression, it also may be used to treat refractive disorders. The agonists, antagonists, or gene therapies employed in this method specifically activates or inactivates key components of the HIPPO pathway, including MST1/MST2 kinase, LATS1/LATS2 kinase, or YAP/TAZ protein.

A particular embodiment of this method involves the use of XMU-MP-1 or its derivative compounds or its pharmaceutically acceptable salt forms as agonists. XMU-MP-1, a benzenesulfonamide recognized for its reversible and selective inhibition of MST1/2, emerges as a potent tool to activate the HIPPO pathway. Administered in dosages ranging from 2 nmol to 200 nmol, XMU-MP-1 demonstrates effectiveness in modulating ocular growth. Importantly, the administration is strategically targeted to traverse the cornea and/or the blood-retinal barrier, ensuring the therapeutic agents reach the retina.

Moreover, the method allows for versatile administration approaches, integrating seamlessly with other delivery systems. These include but are not limited to ultrasonic ocular drug delivery systems, drug-loaded contact lenses, and hydrogel-based delivery mechanisms. By combining the precision of HIPPO pathway targeting with innovative delivery systems, this method represents a comprehensive approach to treating refractive disorders with heightened efficacy and minimized side effects.

“Pharmaceutically acceptable salts” includes derivatives of the compounds, wherein the parent compound is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. The pharmaceutically acceptable salts include non-toxic salts and the quaternary ammonium salts of the parent compound formed, for example, from non-toxic inorganic or organic acids. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts. Pharmaceutically acceptable organic salts includes salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; and combinations comprising one or more of the foregoing salts.

Drug formulation and delivery system are important for ophthalmic applications. The delivery of the agonists, antagonists, or gene therapies targeting HIPPO signaling pathway is necessary to pass through the cornea and/or the blood-retinal barrier and reach the retina. The delivery technologies encompass the following characteristics: (1) easy and noninvasive administration; (2) an efficient delivery system; (3) compatible with ocular tissues; (4) target-specific for the indicated ocular diseases; and (5) a controlled-release system, which keeps active agents for a prolonged period (several months to several years) in the retina.

Sustained-release formulations can be administered once daily or even less frequently. Sustained-release formulations can be based on matrix technology. In this technology, the agent is embedded in an excipient that makes a non-disintegrating core called a matrix. Diffusion of agent occurs through the core.

A pulsed-release dosage form includes an immediate-release dosage form of the agent; and a delayed-release dosage form including the agent.

In one embodiment, a delayed-release dosage form can be combined with an immediate-release dosage form to provide a pulsed-release dosage form. The delayed-release dosage form may be in the form of a core which optionally includes absorption enhancers and/or water swellable substances. Pulsed-release dosage forms allow for control of the plasma levels of the agent.

The term of “immediate-release” means that a conventional or non-modified release form in which greater than or equal to about 75% of the active agent is released within two hours of administration, preferably within one hour of administration.

The term “controlled-release” is a dosage form in which the release of the active agent is controlled or modified over a period of time. Controlled can mean, for example, sustained, delayed or pulsed-release at a particular time. Alternatively, controlled can mean that the release of the active agent is extended for longer than it would be in an immediate-release dosage form, i.e., at least over several hours, such as greater than four hours, preferably greater than eight hours.

The term “sustained-release” or “extended-release” is meant to include the release of the active agent at such a rate that blood (e.g., plasma) levels are maintained within a therapeutic range but below toxic levels for at least about 8 hours, preferably at least about 12 hours after administration at steady-state. The term “steady-state” means that a plasma level for a given active agent has been achieved and which is maintained with subsequent doses of the drug at a level which is at or above the minimum effective therapeutic level and is below the minimum toxic plasma level for a given active agent.

The term “delayed-release” means that there is a time-delay before significant plasma levels of the active agent are achieved. A delayed-release formulation of the active agent can avoid an initial burst of the active agent, or can be formulated so that release of the active agent in eye ball or muscle layer is avoided and absorption takes places in retina.

A “pulsed-release” formulation can contain a combination of immediate-release, sustained-release, and/or delayed-release formulations in the same dosage form. A “semi-delayed-release” formulation is a pulsed-released formulation in which a moderate dosage is provided immediately after administration and a further dosage some hours after administration.

Embarking on a comprehensive approach to treating refractive disorders, the present invention introduces a pharmaceutical composition meticulously designed for optimum efficacy and patient compatibility. This composition comprises an agonist, antagonist, or gene therapy specifically activating or inactivating the HIPPO signaling pathway, strategically formulated with a range of pharmaceutically acceptable additions.

The refractive disorders encompassed by this pharmaceutical composition include myopia and hyperopia, two prevalent conditions that impact vision. The specificity of the treatment is achieved through the targeted action of the agonist, antagonist, or gene therapy on key components of the HIPPO pathway, namely MST1/MST2 kinase, LATS1/LATS2 kinase, or YAP/TAZ protein.

In a particular embodiment, the pharmaceutical composition employs the agonist XMU-MP-1 or its derivative compounds or its pharmaceutically acceptable salt forms. Notably, XMU-MP-1, recognized for its reversible and selective inhibition of MST1/2, assumes a pivotal role in activating the Hippo pathway. Administered in dosages ranging from 2 nmol to 200 nmol, this compound showcases its effectiveness in modulating ocular growth with precision.

The composition is formulated to facilitate targeted delivery to the patient's retina through the cornea and/or the blood-retinal barrier. This strategic formulation is adaptable to various administration forms, including immediate-release or controlled-release forms. Options for administration forms are diverse, covering injection, eye drop, eye ointment, hydrogel, ultrasonic ocular drug delivery, nanoparticle, microemulsion drug delivery, and drug-loaded contact lenses.

Ensuring patient safety and treatment stability, the pharmaceutical composition incorporates a range of pharmaceutically acceptable additions. These include excipients, stability additives, carriers, diluents, and solubilizers, collectively contributing to a pharmaceutical composition poised to revolutionize the landscape of refractive disorder treatments.

In accordance with one embodiment of the present invention, the composition can be formulated as nanoparticle or microemulsion drug delivery systems, which enhances solubility and improve delivery efficiency by surface-conjugating active targeting ligands or improves drug solubilization capacity and bioavailability.

The present invention treats refractive disorders through the use of a pharmaceutical composition. This composition, finely tuned for precision and efficacy, incorporates an agonist, antagonist, or gene therapy specifically designed to specifically activate or inactivate the HIPPO signaling pathway. This approach finds application in subjects affected with refractive disorders, offering a surgery-free approach to treatment.

The refractive disorders in focus encompass the prevalent conditions of myopia or hyperopia, reflecting the versatility of this treatment approach. The specificity and targeted action of the treatment are further underscored by the agonist, antagonist, or gene therapy's unique focus on MST1/MST2 kinase, LATS1/LATS2 kinase, or YAP/TAZ protein.

A particularly impactful embodiment of this approach involves the use of the agonist XMU-MP-1 or its derivative compounds. Renowned for its ability to selectively inhibit MST1/2 while activating the Hippo pathway, this agonist assumes a pivotal role in steering the treatment towards optimal outcomes. Administered at dosages ranging from 2 nmol to 200 nmol, XMU-MP-1 or its derivatives or its pharmaceutically acceptable salt forms establish themselves as potent tools in the quest for refractive disorder treatment.

This use of a pharmaceutical composition, strategically targeting the HIPPO signaling pathway, represents a transformative step in the realm of refractive disorder treatments. Through its specificity, adaptability, and nuanced formulation, this approach aspires to redefine standards in ocular healthcare.

### EXAMPLES

### Example 1. Proteomic Analysis Showing the Involvement of HIPPO Pathway in Regulation of Ocular Growth

In the myopic animal model, concave lenses of-5D are applied to pigmented guinea pigs (Cavia porcellus) at six days old (PN6, baseline), a critical period for rapid eye growth during emmetropization (Howlett and McFadden, 2007). The right eyes are assigned-5D lenses (treated eyes), while the left eyes have no lenses attached (control eyes). After a 7-day Lens-Induced Myopia (LIM) treatment (PN13), the lenses are removed for a 2-day recovery (PN15). Refractive error and ocular parameters are recorded at baseline, 7-day LIM, and 2-day recovery using streak retinoscopy and high-frequency ultrasonography. Each group includes 6 pigmented guinea pigs (12 retinas). Retinal tissues are collected after the 2-day recovery treatment. Despite the 2-day recovery, myopic eyes do not fully compensate to the-5D lens, revealing a significant difference in choroidal thickness (P<0.01, n=6). Relative elongation of axial length and vitreous chamber depth is also observed in myopic eyes (P<0.01, n=6).

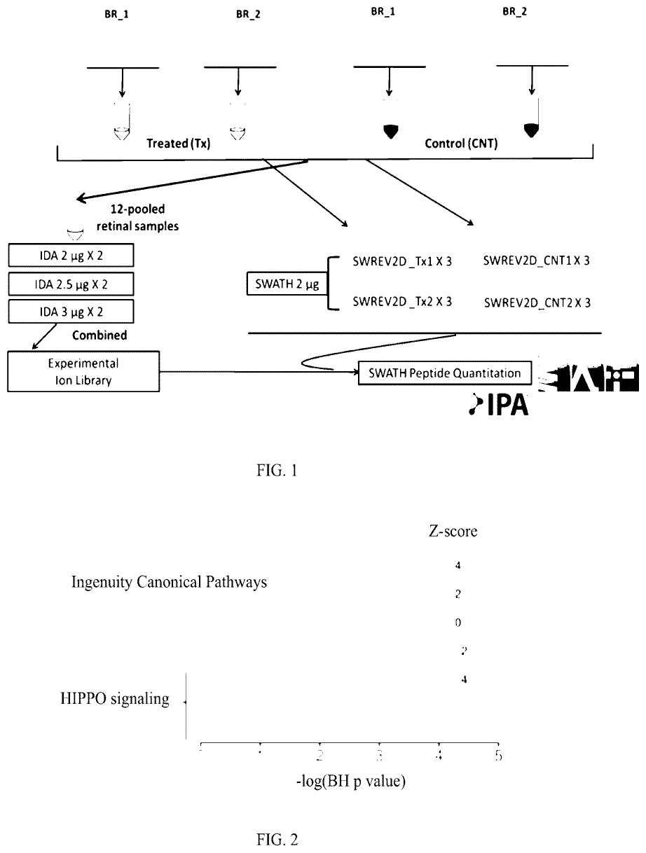

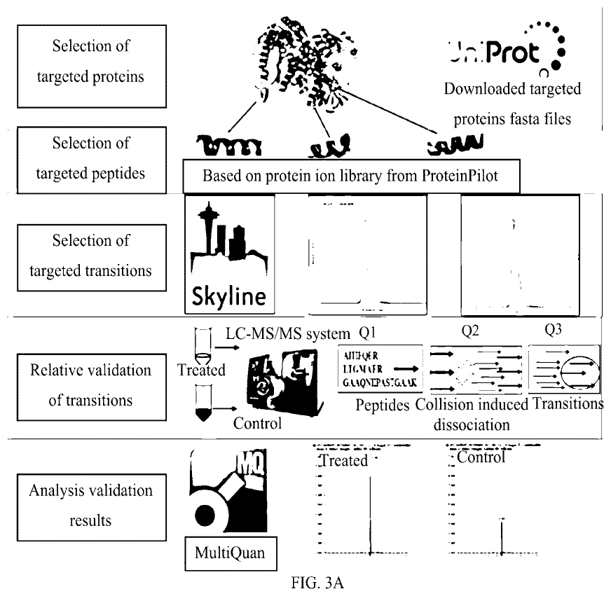

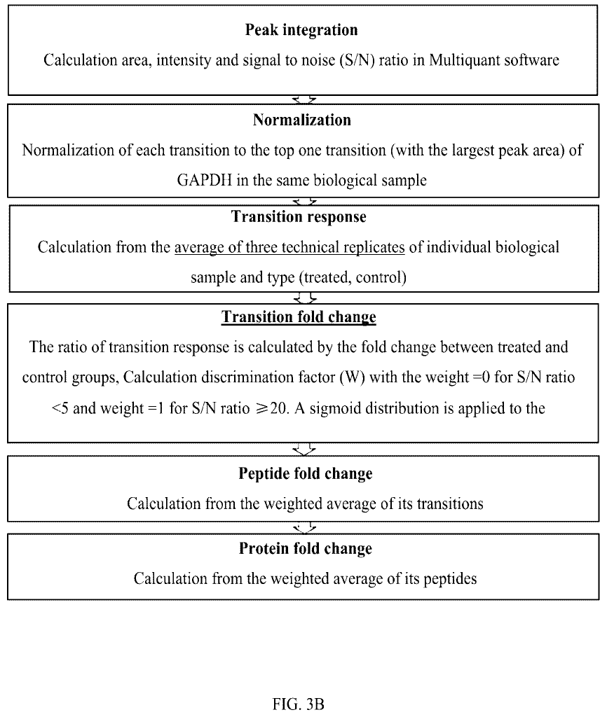

Using a proteomics approach, collected retinal tissues are homogenized, and total protein concentration is measured. Liquid nitrogen-cooled homogenization is performed with 200 μl of customized lysis buffer. The recovered retinal lysates are then centrifuged, and the supernatant is collected. The total protein concentration is measured by Bradford protein assay. Approximately 75 μg of proteins are digested. Differentially expressed proteins during the initial recovery stage are screened using label-free sequential window acquisition of all theoretical mass spectra (SWATH-MS) proteomics (n=6 animals, 12 eyes) by the TripleTOF 6600 system. For protein identification and quantification, a species-specific retinal ion library for SWATH analysis is built, identifying 3579 non-redundant proteins. Seventy-seven up-regulated and 42 down-regulated proteins are found to significantly change after the 2-day recovery treatment (≥1.3 or ≤0.77-fold change, with a similar trend in two biological replicates) (FIG. 1). Ingenuity Pathways Analysis (IPA) indicated the activation of the “HIPPO signaling” pathway associated with the differentially expressed proteins (FIG. 2). Subsequently, individual retinal tissues (n=6) undergo a novel Multiple Reaction Monitoring (MRM) assay for orthogonal protein target confirmation at the second stage by the QTRAP 6500+. The integrated area of individual transition for each peptide is obtained from MultiQuant™ (version 3.03, SCIEX, MA). Based on protein abundance and peptide sequences, five index proteins strongly associated with HIPPO signaling are successfully validated with high confidence (P<0.05) using MRM-based proteomics (FIGS. 3A-3B).

### Example 2. Pharmacologic Testing Showing that Ocular Growth of Animal Eyes Can Be Modulated by Targeting MST1/2 of the HIPPO Pathway

XMU-MP-1, known for its reversible and selective inhibition of MST1/2 (upstream to YAP/TAZ), leading to a significant increase in the activity of the Hippo pathway effector YAP, undergoes testing in a dose-dependent manner for its impact on form-deprivation myopia (FDM) in chicks.

White Leghorn chicks (Gallus gallus) from specific pathogen-free eggs (SPF, Jinan, China) are raised in standard cages with adequate food and water, maintaining a temperature of 25° C. and a 12 h:12 h light/dark cycle. The experimental period spans from postnatal 7 days (PN 7) to 14 days (PN 14). A unilateral FDM model is established using a published protocol, involving four intravitreal injections performed every alternate day. Following the first injection, diffuser lenses are attached to the right eye, with lenses covering only the XMU-MP-1 injected eye (unilateral FDM). XMU-MP- 1, dissolved in 10% DMSO with 90% Corn oil, is injected monocularly using a 0.3 mm (30 G)×8 mm needle into the vitreous through the skin, sclera, choroid, and retina close to the margin of the upper orbit. Contralateral eyes receive no lens treatment. Four concentrations of XMU-MP-1 (0.02, 0.2, 2, and 20 mmol/L) are selected for evaluation.

Animals are randomly assigned to five groups, encompassing the FDM with vehicle treatment group (vehicle group, 10% DMSO+90% Corn oil, n=14), FDM with 0.02 mmol/L XMU-MP-1 treatment group (XMU-MP-1, 0.02 mmol/L group, n=5), FDM with 0.2 mmol/L XMU-MP-1 treatment group (XMU-MP-1, 0.2 mmol/L group, n=5), FDM with 2 mmol/L XMU-MP-1 treatment group (XMU-MP-1, 2 mmol/L group, n=13), and FDM with 20 mmol/L XMU-MP-1 treatment group (XMU-MP-1, 20 mmol/L group, n=14). The experimental groupings do not exhibit a gender preference.

Refractive errors are assessed using a streak retinoscope, and ocular parameters are measured with a high-frequency A-scan ultrasound system equipped with a 30 MHz transducer (Panametrics, Inc., Waltham, MA) before and after treatment. The spherical equivalent (S.E.) is derived from the refractive status (S.E.=spherical power+½ cylindrical power), and axial length is defined from the front of the cornea to the back of the vitreous chamber.

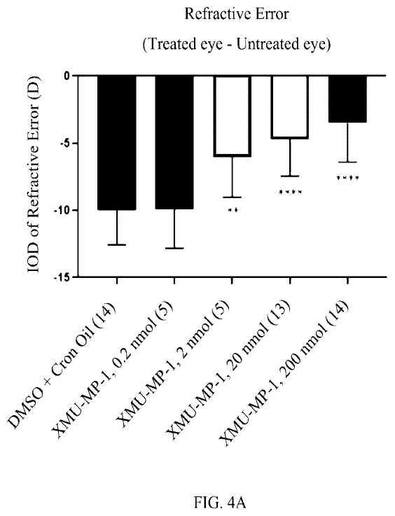

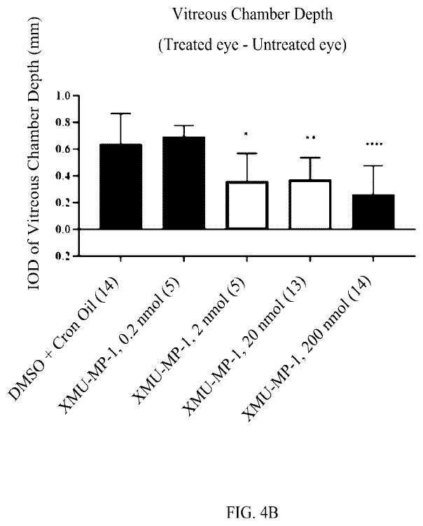

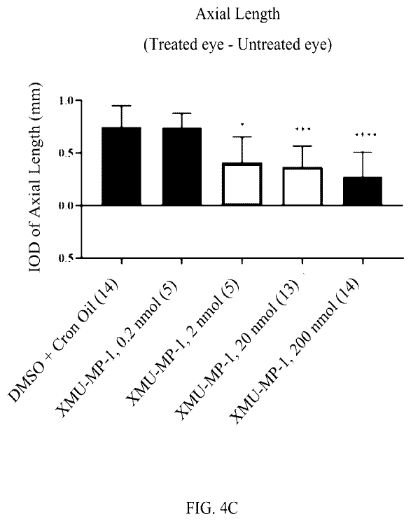

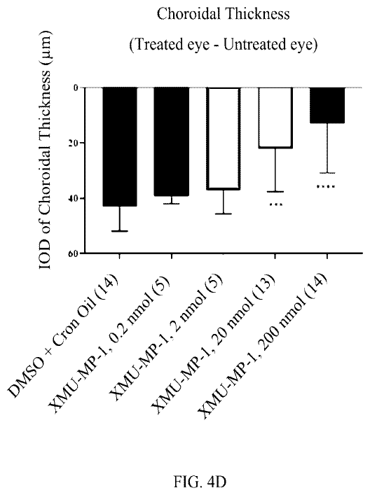

For statistical analysis, after the 7-day lens treatment period, interocular differences (the difference between the diffuser-treated eyes and the contralateral fellow eyes) are calculated for six ocular parameters (refractive error, anterior chamber depth, lens thickness, vitreous chamber depth (FIG. 4B), axial length, and choroidal thickness). Mean interocular differences±SD are presented, and the significance of the dose-effect is analyzed using ANOVA. Post hoc testing (Dunnett's method) is employed where appropriate, with significance levels denoted as follows: ns, not significant; *, P<0.05; **, P<0.01; ****, P<0.0001.

In unilateral FDM chicks, no significant differences are observed in all ocular parameters among the various groups at baseline. As shown in FIG. 4A, following a 7-day treatment, myopic progression is more pronounced in the vehicle group (−10.0±2.56 D) compared to the 0.2 mmol/L (−6.05±2.97 D, P<0.01), 2 mmol/L (−4.69±2.75, P<0.0001), and 20 mmol/L groups (−3.51±2.89 D, P<0.0001). As shown in FIG. 4C, axial length is also greater in the vehicle group (0.75±0.19 mm) and 0.02 mmol/L (0.73±0.13 mm) groups, compared to 0.2 mmol/L (0.41±0.24 mm, P<0.001), 2 mmol/L (0.36±0.20 mm, P<0.001), and 20 mmol/L groups (0.27±0.22 mm, P<0.0001). Additionally, as shown in FIG. 4D, XMU-MP-1 induces an increase in choroidal thickness by about 50% and 70% in 2 mmol/L (22.07±15.13 μm, P<0.001) and 20 mmol/L groups (12.83±17.96 μm, P<0.0001) respectively, compared to the vehicle group (43.02±8.87 μm). In summary, XMU-MP-1 can suppress myopia progression through intravitreal injection in a dose-dependent manner (ANOVA: significant dose-response effect (p<0.0001)). Based on refractive error and axial length data, about 40% inhibition occurred in the 0.2 mmol/L group, and about 53% inhibition in the 2 mmol/L group. Myopia development is suppressed about 65% at the highest dose tested (200 nmol). High myopia inhibition is shown in biometrics, including refractive errors, axial length, and choroidal thickness change.

While the precise mechanism of the existing drug “atropine” remains unclear, the HIPPO signaling pathway has been successfully screened and validated through a combination of SWATH and MRM-based proteomics approaches. The SWATH-MS-based proteomics approach has gained popularity for biomarker discovery and understanding biological mechanisms due to its high reproducibility, resolution, and speed of protein detection. In targeted-based proteomics, multiple reaction monitoring (MRM) enables the quantification of multiple proteins of interest in a single experiment, providing higher throughput, sensitivity, and efficiency compared to immune-detection methods. The invention is rooted in the application of more accurate and reliable techniques.

XMU-MP1 has demonstrated high effectiveness and, importantly, no signs of toxicity in animal models. The HIPPO signaling pathway, an emerging biological pathway, is attracting attention from pharmaceutical companies due to its role in systemic diseases and cancers. Ongoing exploration of XMU-MP-1 effects in myopic mammalian models is part of this endeavor.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A method of treating a refractive disorder, comprising administering an agonist, an antagonist or a gene therapy selectively activates or inactivates the HIPPO signaling pathway, a signaling pathway influences cellular proliferation, organ growth and organ regeneration; wherein the administration is through the cornea and/or the blood-retinal barrier and reach the retina.

2. The method of claim 1, wherein the refractive disorder comprises myopia and hyperopia.

3. The method of claim 1, wherein the agonist, the antagonist or the gene therapy specifically targets MST1/MST2 kinase, LATS1/LATS2 kinase or YAP/TAZ protein.

4. The method of claim 3, wherein the agonist comprises XMU-MP-1, a pharmaceutically acceptable salt thereof or a derivative compound thereof.

5. The method of claim 4, wherein the XMU-MP-1 or the derivative compound thereof inhibits MST1/2 and activates the HIPPO pathway.

6. The method of claim 4, wherein the XMU-MP-1 or the derivative compound thereof is in a dosage of 2 nmol to 200 nmol.

7. The method of claim 1, wherein the administration is in an administration form comprising an injection form, an eye drop form, an eye ointment form, a hydrogel form, an ultrasonic ocular drug delivery form, a nanoparticle form, a microemulsion drug delivery form, and a drug-loaded contact lenses form; and wherein the administration is integrated with other delivery systems comprising ultrasonic ocular drug delivery systems, drug-loaded contact lenses, and hydrogel.

8. (canceled)

9. A pharmaceutical composition comprising an agonist, an antagonist or a gene therapy selectively activating or inactivating the HIPPO signaling pathway, a signaling pathway influences cellular proliferation, organ growth and organ regeneration, for treating a refractive disorder in a subject in need thereof, further comprising a pharmaceutically acceptable addition; wherein the composition is formulated to an administration form of that enables delivery to the patient's retina through the cornea and/or the blood-retinal barrier.

10. The pharmaceutical composition of claim 9, wherein the refractive disorder comprises myopia and hyperopia.

11. The pharmaceutical composition of claim 9, wherein the agonist, the antagonist or the gene therapy specifically targets MST1/MST2 kinase, LATS1/LATS2 kinase or YAP/TAZ protein.

12. The pharmaceutical composition of claim 11, wherein the agonist comprises XMU-MP-1, a pharmaceutically acceptable salt thereof or a derivative compound thereof.

13. The pharmaceutical composition of claim 12, wherein the XMU-MP-1 or the derivative compound thereof inhibits MST1/2 and activate the Hippo pathway.

14. The pharmaceutical composition of claim 12, wherein the XMU-MP-1 or the derivative compound thereof is in a dosage of 2 nmol to 200 nmol.

15. (canceled)

16. The pharmaceutical composition of claim 9, wherein the administration form is selected from an immediate-release form or a controlled-release form.

17. The pharmaceutical composition of claim 16, wherein the administration form comprises an injection form, an eye drop form, an eye ointment form, a hydrogel form, an ultrasonic ocular drug delivery form, a nanoparticle form, a microemulsion drug delivery form, and a drug-loaded contact lenses form.

18. The pharmaceutical composition of claim 9, wherein the pharmaceutically acceptable addition comprises an excipient, a stability additive, a carrier, a diluent, and a solubilizer.

19. Use of the pharmaceutical composition of claim 9 for treating a refractive disorder in a subject in need thereof, comprising: delivering the pharmaceutical composition to through the cornea and/or the blood-retinal barrier and reach the subject's retina.

20. The use of claim 19, wherein the refractive disorder is selected from myopia or hyperopia.

21. The use of claim 19, wherein the agonist, the antagonist or the gene therapy specifically targets MST1/MST2 kinase, LATS1/LATS2 kinase or YAP/TAZ protein.

22. The use of claim 21, wherein the agonist comprises XMU-MP-1, a pharmaceutically acceptable salt thereof or a derivative compound thereof.

23. (canceled)

24. (canceled)
